(19) United States
(12) Patent Application Publication
(10) Pub. No.: US 2023/0393544 A1
(43) Pub. Date: Dec. 7, 2023

(54) TECHNIQUES FOR ADJUSTING A HEADREST OF A COMPUTER-ASSISTED SYSTEM

(71) Applicant: INTUITIVE SURGICAL OPERATIONS, INC., Sunnyvale, CA (US)

(72) Inventors: Ehsan NOOHI BEZANJANI, Los Gatos, CA (US); Lawton N. VERNER, Saratoga, CA (US)

(21) Appl. No.: 18/326,567

(22) Filed: May 31, 2023

Related U.S. Application Data
(60) Provisional application No. 63/347,964, filed on Jun. 1, 2022.

(51) Int. Cl. G05B 15/02

(52) U.S. Cl. CPC G05B 15/02; A61B 90/37

(57) ABSTRACT

Techniques for adjusting a headrest of a computer-assisted system include the following. The computer-assisted system includes a display unit configured to display images viewable by an operator, a headrest coupled to the display unit, the headrest configured to be contacted by a head of the operator, an actuator operable to move the headrest relative to the display unit, a head-input sensor, and a control unit communicably coupled to the actuator and the head-input sensor. The control unit is configured to: determine head data based on sensor data acquired by the head-input sensor, determine a commanded motion based on at least the head data, a baseline, and a damping, and command the actuator to move the headrest based on the commanded motion.

### RELATED APPLICATION

This application claims priority to U.S. Provisional Patent Application No. 63/347,964, filed Jun. 1, 2022 and titled “Techniques for Adjusting a Headrest of a Computer-Assisted System,” which is incorporated by reference herein.

### TECHNICAL FIELD

The present disclosure relates generally to electronic devices and more particularly relates to adjusting a headrest of a computer-assisted system.

### BACKGROUND

Computer-assisted electronic systems are being used more and more often. This is especially true in industrial, entertainment, educational, and other settings. As a medical example, the medical facilities of today have large arrays of electronic devices being found in operating rooms, interventional suites, intensive care wards, emergency rooms, and/or the like. Many of these electronic devices may be capable of autonomous or semi-autonomous motion. It is also known for personnel to control the motion and/or operation of electronic devices using one or more input devices located at a user control system. As a specific example, minimally invasive, robotic telesurgical systems permit surgeons to operate on patients from bedside or remote locations. Telesurgery refers generally to surgery performed using surgical systems where the surgeon uses some form of remote control, such as a servomechanism, to manipulate surgical instrument movements rather than directly holding and moving the instruments by hand.

When an electronic device is used to perform a task at a worksite, one or more imaging devices (e.g., an endoscope) can capture images of the worksite that provide visual feedback to an operator who is monitoring and/or performing the task. The imaging device(s) may be controllable to update a view of the worksite that is provided, such as via a display unit, to the operator. The display unit may have lenses and/or view screens.

To use the display unit, the operator can position his or her head against a headrest of the display unit so as to view images displayed on one or more view screens of the display unit, either directly or through one or more intervening components. However, when the headrest is poorly adjusted, the operator can experience discomfort, unsatisfactory views of images being displayed, stereoscopic images that do not properly fuse, etc. As a result, the operator may experience frustration, eye fatigue, inaccurate depictions of the items in the images, etc.

Accordingly, improved techniques for adjusting a headrest of a computer-assisted system are desirable.

### SUMMARY

Consistent with some embodiments, a computer-assisted system includes a display unit configured to display images viewable by an operator, a headrest coupled to the display unit, the headrest configured to be contacted by a head of the operator, an actuator operable to move the headrest relative to the display unit, a head-input sensor, and a control unit communicably coupled to the actuator and the head-input sensor. The control unit is configured to: determine head data based on sensor data acquired by the head-input sensor, determine a commanded motion based on at least the head data, system data, a baseline, and a damping, and command the actuator to move the headrest based on the commanded motion.

Other embodiments include, without limitation, one or more non-transitory machine-readable media including a plurality of machine-readable instructions, which when executed by one or more processors, are adapted to cause the one or more processors to perform any of the methods disclosed herein.

The foregoing general description and the following detailed description are exemplary and explanatory in nature and are intended to provide an understanding of the present disclosure without limiting the scope of the present disclosure. In that regard, additional aspects, features, and advantages of the present disclosure will be apparent to one skilled in the art from the following detailed description.

### BRIEF DESCRIPTION OF THE DRAWINGS

FIG. 1 is a simplified diagram including an example of a computer-assisted system, according to various embodiments.

FIG. 2 is a perspective view of an example display system, according to various embodiments.

FIG. 3 illustrates the control module of FIG. 1 in greater detail, according to various embodiments.

FIG. 4 illustrates a simplified diagram of a method for adjusting a headrest of a computer-assisted system in a linear degree of freedom (DOF), according to various embodiments.

FIG. 5 illustrates a simplified diagram of a method for adjusting a headrest of a computer-assisted system in a rotational DOF, according to various embodiments.

FIG. 6 illustrates an example of controlling a headrest of a display unit, according to various embodiments.

### DETAILED DESCRIPTION

This description and the accompanying drawings that illustrate inventive aspects, embodiments, embodiments, or modules should not be taken as limiting—the claims define the protected invention. Various mechanical, compositional, structural, electrical, and operational changes may be made without departing from the spirit and scope of this description and the claims. In some instances, well-known circuits, structures, or techniques have not been shown or described in detail in order not to obscure the invention. Like numbers in two or more figures represent the same or similar elements.

In this description, specific details are set forth describing some embodiments consistent with the present disclosure. Numerous specific details are set forth in order to provide a thorough understanding of the embodiments. It will be apparent, however, to one skilled in the art that some embodiments may be practiced without some or all of these specific details. The specific embodiments disclosed herein are meant to be illustrative but not limiting. One skilled in the art may realize other elements that, although not specifically described here, are within the scope and the spirit of this disclosure. In addition, to avoid unnecessary repetition, one or more features shown and described in association with one embodiment may be incorporated into other embodiments unless specifically described otherwise or if the one or more features would make an embodiment non-functional.

Further, the terminology in this description is not intended to limit the invention. For example, spatially relative terms-such as “beneath”, “below”, “lower”, “above”, “upper”, “proximal”, “distal”, and the like—may be used to describe one element's or feature's relationship to another element or feature as illustrated in the figures. These spatially relative terms are intended to encompass different positions (i.e., locations) and orientations (i.e., rotational placements) of the elements or their operation in addition to the position and orientation shown in the figures. For example, if the content of one of the figures is turned over, elements described as “below” or “beneath” other elements or features would then be “above” or “over” the other elements or features. Thus, the exemplary term “below” can encompass both positions and orientations of above and below. A device may be otherwise oriented (rotated 90 degrees or at other orientations) and the spatially relative descriptors used herein interpreted accordingly. Likewise, descriptions of movement along and around various axes include various special element positions and orientations. In addition, the singular forms “a”, “an”, and “the” are intended to include the plural forms as well, unless the context indicates otherwise. And, the terms “comprises”, “comprising”, “includes”, and the like specify the presence of stated features, steps, operations, elements, and/or components but do not preclude the presence or addition of one or more other features, steps, operations, elements, components, and/or groups. Components described as coupled may be electrically or mechanically directly coupled, or they may be indirectly coupled via one or more intermediate components.

Elements described in detail with reference to one embodiment, embodiment, or module may, whenever practical, be included in other embodiments, embodiments, or modules in which they are not specifically shown or described. For example, if an element is described in detail with reference to one embodiment and is not described with reference to a second embodiment, the element may nevertheless be claimed as included in the second embodiment. Thus, to avoid unnecessary repetition in the following description, one or more elements shown and described in association with one embodiment, embodiment, or application may be incorporated into other embodiments, embodiments, or aspects unless specifically described otherwise, unless the one or more elements would make an embodiment or embodiment non-functional, or unless two or more of the elements provide conflicting functions.

In some instances, well known methods, procedures, components, and circuits have not been described in detail so as not to unnecessarily obscure aspects of the embodiments.

This disclosure describes various devices, elements, and portions of computer-assisted systems and elements in terms of their state in three-dimensional space, often described with three translational degrees of freedom and three rotational degrees of freedom. It is understood, however, that in instances where one or more translational or rotational degrees of freedom are insignificant for a particular feature, such feature may operate with full three-dimensional spatial information about physical elements, or with lower dimensional information with fewer degrees of freedom. As used herein, and for a device with a kinematic chain such as a repositionable structure comprising a manipulator arm, the term “proximal” refers to a direction toward the base of the device along the kinematic chain and “distal” refers to a direction away from the base along the kinematic chain.

Aspects of this disclosure are described in reference to computer-assisted systems and devices, which may include systems and devices that are teleoperated, remote-controlled, autonomous, semiautonomous, robotic, and/or the like. Further, aspects of this disclosure are described in terms of an embodiment using a medical system, such as the da Vinci® Surgical System commercialized by Intuitive Surgical, Inc. of Sunnyvale, California. Knowledgeable persons will understand, however, that inventive aspects disclosed herein may be embodied and implemented in various ways, including robotic and, if applicable, non-robotic embodiments. Embodiments described for da Vinci® Surgical Systems are merely exemplary, and are not to be considered as limiting the scope of the inventive aspects disclosed herein. For example, techniques described with reference to surgical instruments and surgical methods may be used in other contexts. Thus, the instruments, systems, and methods described herein may be used for humans, animals, portions of human or animal anatomy, industrial systems, general robotic, or teleoperational systems. As further examples, the instruments, systems, and methods described herein may be used for non-medical purposes including industrial uses, general robotic uses, sensing or manipulating non-tissue work pieces, cosmetic improvements, imaging of human or animal anatomy, gathering data from human or animal anatomy, setting up or taking down systems, training medical or non-medical personnel, and/or the like. Additional example applications include use for procedures on tissue removed from human or animal anatomies (with or without return to a human or animal anatomy) and for procedures on human or animal cadavers. Further, these techniques can also be used for medical treatment or diagnosis procedures that include, or do not include, surgical aspects.

### System Overview

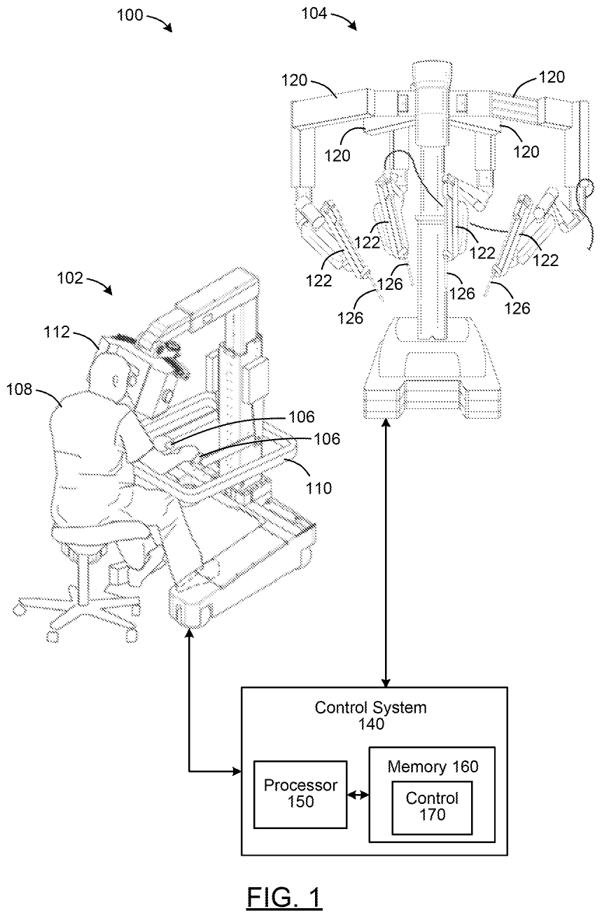

FIG. 1 is a simplified diagram of an example computer-assisted system, according to various embodiments. In some examples, the computer-assisted system is a teleoperated system 100. In medical examples, teleoperated system 100 can be a teleoperated medical system such as a surgical system. As shown, teleoperated system 100 includes a follower device 104 that may be teleoperated by being controlled by one or more leader devices (also called “leader input devices” when designed to accept external input), described in greater detail below. Systems that include a leader device and a follower device are referred to as leader-follower systems, and also sometimes referred to as master-slave systems. Also shown in FIG. 1 is an input system that includes a workstation 102 (e.g., a console), and in various embodiments the input system can be in any appropriate form and may or may not include a workstation 102.

In the example of FIG. 1, workstation 102 includes one or more leader input devices 106 which are designed to be contacted and manipulated by an operator 108. For example, workstation 102 can comprise one or more leader input devices 106 for use by the hands, the head, or some other body part of operator 108. Leader input devices 106 in this example are supported by workstation 102 and can be mechanically grounded. In some embodiments, an ergonomic support 110 (e.g., forearm rest) can be provided on which the operator 108 can rest his or her forearms. In some examples, the operator 108 can perform tasks at a worksite near the follower device 104 during a procedure by commanding follower device 104 using leader input devices 106.

A display unit 112 is also included in the workstation 102. The display unit 112 can display images for viewing by the operator 108. The display unit 112 can be moved in various degrees of freedom to accommodate the viewing position of the operator 108 and/or to optionally provide control functions as another leader input device. In the example of the teleoperated system 100, displayed images can depict a worksite at which the operator 108 is performing various tasks by manipulating the leader input devices 106 and/or the display unit 112. In some examples, the images displayed by the display unit 112 can be received by the workstation 102 from one or more imaging devices arranged at the worksite. In other examples, the images displayed by the display unit 112 can be generated by the display unit 112 (or by a different connected device or system), such as for virtual representations of tools, the worksite, or for user interface components.

When using the workstation 102, the operator 108 can sit in a chair or other support in front of the workstation 102, position his or her eyes in front of the display unit 112, manipulate the leader input devices 106, and rest his or her forearms on the ergonomic support 110 as desired. In some embodiments, the operator 108 can stand at the workstation or assume other poses, and the display unit 112 and leader input devices 106 can be adjusted in position (height, depth, etc.) to accommodate the operator 108.

Teleoperated system 100 can also include follower device 104, which can be commanded by workstation 102. In a medical example, follower device 104 can be located near an operating table (e.g., a table, bed, or other support) on which a patient can be positioned. In some medical examples, the worksite is provided on an operating table, e.g., on or in a patient, simulated patient, or model, etc. (not shown). The follower device 104 shown includes a plurality of manipulator arms 120, each manipulator arm 120 configured to couple to an instrument assembly 122. An instrument assembly 122 can include, for example, an instrument 126.

In various embodiments, one or more of the instruments 126 can include an imaging device for capturing images (e.g., optical cameras, hyperspectral cameras, ultrasonic sensors, etc.). For example, one or more of the instruments 126 could be an endoscope assembly that includes an imaging device, which can provide captured images of a portion of the worksite to be displayed via the display unit 112.

In some embodiments, the manipulator arms 120 and/or instrument assemblies 122 can be controlled to move and articulate instruments 126 in response to manipulation of leader input devices 106 by operator 108, and in this way “follow” through teleoperation the leader input devices 106. This enables the operator 108 to perform tasks at the worksite using the manipulator arms 120 and/or instrument assemblies 122. The manipulator arms 120 and instrument assemblies 122 are examples of repositionable structures on which instruments and/or imaging devices can be mounted. The repositionable structure(s) of a computer-assisted system comprise the repositionable structure system of the computer-assisted system. For a surgical example, the operator could direct the follower manipulator arms 120 to move instruments 126 to perform surgical procedures at internal surgical sites through minimally invasive apertures or natural orifices.

As shown, a control system 140 is provided external to the workstation 102 and communicates with the workstation 102. In other embodiments, the control system 140 can be provided in the workstation 102 or in the follower device 104. As the operator 108 moves leader input device(s) 106, sensed spatial information including sensed position and/or orientation information is provided to the control system 140 based on the movement of the leader input devices 106. The control system 140 can determine or provide control signals to the follower device 104 to control the movement of the manipulator arms 120, instrument assemblies 122, and/or instruments 126 based on the received information and operator input. In one embodiment, the control system 140 supports one or more wired communication protocols, (e.g., Ethernet, USB, and/or the like) and/or one or more wireless communication protocols (e.g., Bluetooth, IrDA, HomeRF, IEEE 1002.11, DECT, Wireless Telemetry, and/or the like).

The control system 140 can be implemented on one or more computing systems. The one or more computing systems can be used to control the follower device 104. In addition, one or more computing systems can be used to control components of the workstation 102, such as movement of a display unit 112.

As shown, the control system 140 includes a processor 150 and a memory 160 storing a control module 170. In some embodiments, the control system 140 can include one or more processors, non-persistent storage (e.g., volatile memory, such as random access memory (RAM), cache memory), persistent storage (e.g., a hard disk, an optical drive such as a compact disk (CD) drive or digital versatile disk (DVD) drive, a flash memory, etc.), a communication interface (e.g., Bluetooth interface, infrared interface, network interface, optical interface, etc.), and numerous other elements and functionalities. In addition, functionality of the control module 170 can be implemented in any technically feasible software and/or hardware.

Each of the one or more processors of the control system 140 can be an integrated circuit for processing instructions. For example, the one or more processors can be one or more cores or micro-cores of a processor, a central processing unit (CPU), a microprocessor, a field-programmable gate array (FPGA), an application-specific integrated circuit (ASIC), a digital signal processor (DSP), a graphics processing unit (GPU), a tensor processing unit (TPU), and/or the like. The control system 140 can also include one or more input devices, such as a touchscreen, keyboard, mouse, microphone, touchpad, electronic pen, or any other type of input device.

A communication interface of the control system 140 can include an integrated circuit for connecting the computing system to a network (not shown) (e.g., a local area network (LAN), a wide area network (WAN) such as the Internet, mobile network, or any other type of network) and/or to another device, such as another computing system.

Further, the control system 140 can include one or more output devices, such as a display device (e.g., a liquid crystal display (LCD), a plasma display, touchscreen, organic LED display (OLED), projector, or other display device), a printer, a speaker, external storage, or any other output device. One or more of the output devices can be the same or different from the input device(s). Many different types of computing systems exist, and the aforementioned input and output device(s) can take other forms.

In some embodiments, the control system 140 can be connected to or be a part of a network. The network can include multiple nodes. The control system 140 can be implemented on one node or on a group of nodes. By way of example, the control system 140 can be implemented on a node of a distributed system that is connected to other nodes. By way of another example, the control system 140 can be implemented on a distributed computing system having multiple nodes, where different functions and/or components of the control system 140 can be located on a different node within the distributed computing system. Further, one or more elements of the aforementioned control system 140 can be located at a remote location and connected to the other elements over a network.

Software instructions in the form of computer readable program code to perform embodiments of the disclosure can be stored, in whole or in part, temporarily or permanently, on a non-transitory computer readable medium such as a CD, DVD, storage device, a diskette, a tape, flash memory, physical memory, or any other computer readable storage medium. Specifically, the software instructions can correspond to computer readable program code that, when executed by a processor(s) (e.g., processor 150), is configured to perform some embodiments of the methods described herein.

In some embodiments, the one or more leader input devices 106 can be ungrounded (ungrounded leader input devices being not kinematically grounded, such as leader input devices held by the hands of the operator 108 without additional physical support). Such ungrounded leader input devices can be used in conjunction with the display unit 112. In some embodiments, the operator 108 can use a display unit 112 positioned near the worksite, such that the operator 108 manually operates instruments at the worksite, such as a laparoscopic instrument in a surgical example, while viewing images displayed by the display unit 112.

Some embodiments can include one or more components of a teleoperated medical system such as a da Vinci® Surgical System, commercialized by Intuitive Surgical, Inc. of Sunnyvale, California, U.S.A. Embodiments on da Vinci® Surgical Systems are merely examples and are not to be considered as limiting the scope of the features disclosed herein. For example, different types of teleoperated systems having follower devices at worksites, as well as non-teleoperated systems, can make use of features described herein.

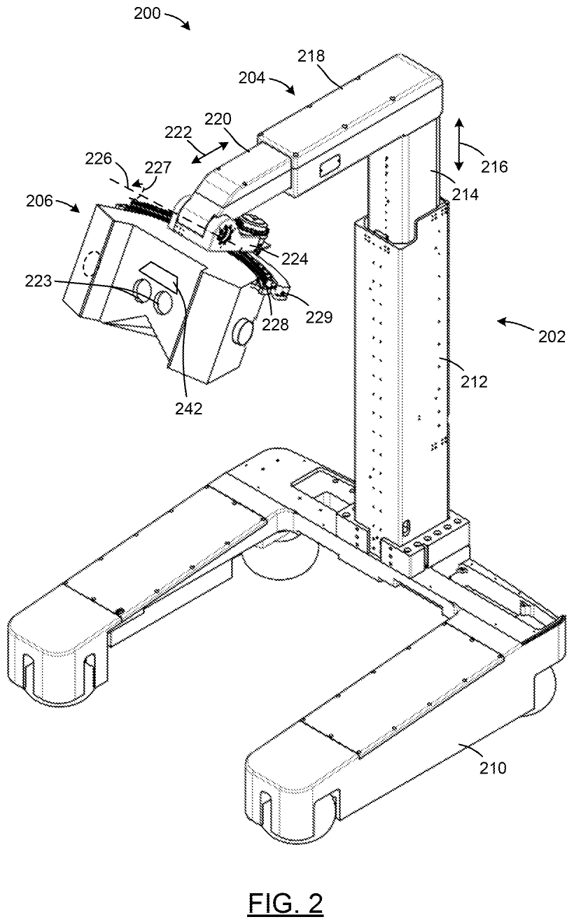

FIG. 2 is a perspective view of an example display system 200 of a computer-assisted system, according to various embodiments. In some embodiments, the display system 200 is used in a workstation of a teleoperated system (e.g., in workstation 102 of the teleoperated system 100 of FIG. 1), or the display system 200 can be used in other systems or as a standalone system, e.g., to allow an operator to view a worksite or other physical site, a displayed virtual environment, etc. Although FIG. 2 shows specific configurations of the display system 200, other embodiments may use different configurations.

As shown in FIG. 2, the display system 200 includes a base support 202, an arm support 204, and a display unit 206. The display unit 206 is provided with multiple degrees of freedom of movement provided by a support linkage including base support 202, an arm support 204 coupled to the base support 202, and a tilt member 224 (described below) coupled to the arm support 204, where the display unit 206 is coupled to the tilt member 224.

The base support 202 can be a vertical member that is mechanically grounded, e.g., directly or indirectly coupled to ground, such as by resting or being attached to a floor. For example, the base support 202 can be mechanically coupled to a wheeled support structure 210 that is coupled to the ground. The base support 202 includes a first base portion 212 and a second base portion 214 coupled such that the second base portion 214 is translatable with respect to the first base portion 212 in a linear degree of freedom 216.

The arm support 204 can be a horizontal member that is mechanically coupled to the base support 202. The arm support 204 includes a first arm portion 218 and a second arm portion 220. The second arm portion 220 is coupled to the first arm portion 218 such that the second arm portion 220 is linearly translatable in a first linear DOF 222 with respect to the first arm portion 218.

The display unit 206 can be mechanically coupled to the arm support 204. The display unit 206 can be moveable in other linear DOFs provided by the linear translations of the second base portion 214 and the second arm portion 220.

In some embodiments, the display unit 206 includes a display, e.g., one or more display screens, projectors, or the like that can display digitized images. In the example shown, the display unit 206 further includes lenses 223 that provide viewports through which the display device can be viewed. As used herein, “lenses” refers to a single lens or multiple lenses, such as a separate lens for each eye of an operator, and “eyes” refers to a single eye or both eyes of an operator. Any technically feasible lenses can be used in embodiments, such as lenses having high optical power. Although display units that include lenses, through which images are viewed, are described herein as a reference example, some embodiments of display units may not include such lenses. For example, in some embodiments, the images displayed by a display unit can be viewed via an opening that allows the viewing of displayed images, viewed directly as displayed by a display screen of the display unit, or in any other technically feasible manner.

In some embodiments, the display unit 206 displays images of a worksite (e.g., an interior anatomy of a patient in a medical example), captured by one or more imaging devices, such as an endoscope. The images can alternatively depict a virtual representation of a worksite that is computer-generated. The images can show captured images or virtual renderings of instruments 126 of the follower device 104 while one or more of these instruments 126 are controlled by the operator via the leader input devices (e.g., the leader input devices 106 and/or the display unit 206) of the workstation 102.

In some embodiments, the display unit 206 is rotationally coupled to the arm support 204 by a tilt member 224. In the illustrated example, the tilt member 224 is coupled at a first end to the second arm portion 220 of the arm support 204 by a rotary coupling configured to provide rotational motion of the tilt member 224 and the display unit 206 about the tilt axis 226 with respect to the second arm portion 220.

Each of the various degrees of freedom discussed herein can be passive and require manual manipulation for movement, or be movable by one or more actuators, such as by one or more motors, solenoids, etc. For example, the rotational motion of the tilt member 224 and the display unit 206 about the tilt axis 226 can be driven by one or more actuators, such as by a motor coupled to the tilt member at or near the tilt axis 226.

The display unit 206 can be rotationally coupled to the tilt member 224 and can rotate about a yaw axis. For example, the rotation can be a lateral or left-right rotation from the point of view of an operator viewing images displayed by the display unit 206. In this example, the display unit 206 is coupled to the tilt member by a rotary mechanism which can comprise a track mechanism that constrains the motion of the display unit 206. For example, in some embodiments, the track mechanism includes a curved member 228 that slidably engages a track 229, thus allowing the display unit 206 to rotate about a yaw axis by moving the curved member 228 along the track 229.

The display system 200 can thus provide the display unit 206 with a vertical linear degree of freedom 216, a horizontal linear DOF 222, and a rotational (tilt) degree of freedom 227. A combination of coordinated movement of components of the display system 200 in these degrees of freedom allows the display unit 206 to be positioned at various positions and orientations based on the preferences of an operator. The motion of the display unit 206 in the tilt, horizontal, and vertical degrees of freedom allow the display unit 206 to stay close to, or maintain contact with, the head of the operator.

Illustratively, the display unit 206 is coupled to a headrest 242. The headrest 242 can be separate from, or integrated within the display unit 206, in various embodiments. In some embodiments, the headrest 242 is coupled to a surface of the display unit 206 that is facing the head of the operator during operation of the display unit 206. The headrest 242 is configured to be able to contact the head of the operator, such as a forehead of the operator, while the operator is viewing images that are displayed via the display unit 206. In some embodiments, the headrest 242 can include one or more head-input sensors that sense inputs applied to the headrest 242 or the display unit 206 in a region above the lenses 223. In such cases, each head-input sensor can include any of a variety of types of sensors, e.g., resistance sensors, capacitive sensors, force sensors, optical sensors, etc. In some other embodiments, one or more head-input sensors can be disposed at any technically feasible location or locations. For example, in some embodiments, one or more head-input sensors can be mounted on the display unit 206. In some embodiments, the headrest 242 is physically coupled to one or more actuators that can be actuated to move the headrest 242 in any number of DOFs, including six DOFs.

It is understood that FIG. 2 merely shows an example for a configuration of a display system. Alternative configurations supporting movement of the display unit 206 and/or the headrest 242 based on operator input are also possible.

### Adjusting a Headrest of a Computer-Assisted System

The headrest of a computer-assisted system can be adjusted to reposition a geometric relationship of the eye(s) of an operator relative to image(s) displayed by a display unit based on linear and/or rotational inputs that are sensed by one or more head-input sensors and linear and/or rotational data that is generated by a control module.

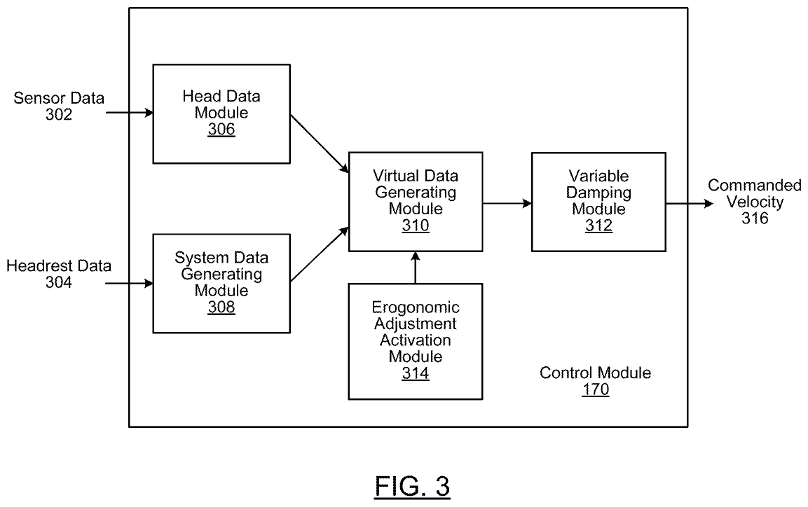

FIG. 3 illustrates the control module 170 of FIG. 1 in greater detail, according to various embodiments. As shown, the control module 170 includes a head data module 306, a system data generating module 308, a virtual data generating module 310, a variable damping module 312, and an ergonomic adjustment activation module 314. The head data module 306 generates head input data (also referred to herein as “head data”) based on sensor data 302. In some embodiments, the head data is generated by disregarding (such as by setting to zero sensor data entries) any portions of the sensor data 302 inconsistent with expected head input. Example inconsistent portions in some instances comprise data that correspond to directions in which the head of an operator cannot, or would not, move a headrest during normal operation. For example, in some embodiments, the physical design of the headrest and display unit are such that a head of an operator can only apply a force that pushes the headrest in an “inward” direction towards the display unit, and cannot provide a force that pulls the headrest in an “outward” direction away from the display unit. In such cases, the head data module 306 may be configured to set to zero or a default value, remove, not pass along, or otherwise disregard portions of the sensor data 302 that correspond to “outward” head forces.

In some embodiments, the sensor data 302 and the head data can be in any number of DOFs, including six or fewer spatial DOFs where information in three-dimensional space is sent or determined. In addition, the sensor data 302 and the head data can be represented as vectors, matrices, or in any other suitable manner. The sensor data 302 can be acquired via one or more head-input sensors that sense linear inputs (e.g., forces) or positions or translations, and/or rotational inputs (e.g., torques) and/or orientations or rotations of the head of an operator. For example, in some embodiments, the sensor data 302 can be acquired by one or more of the head-input sensors described above in conjunction with FIG. 2. In addition, the one or more head-input sensors can include strain gauges, touch sensors, springs, one or more time-of-flight (TOF) sensors (e.g., an array of TOF sensors), a combination thereof, etc. in some embodiments. In some embodiments, sensor data corresponding to DOFs in which a headrest cannot be adjusted are discarded by the control module 170.

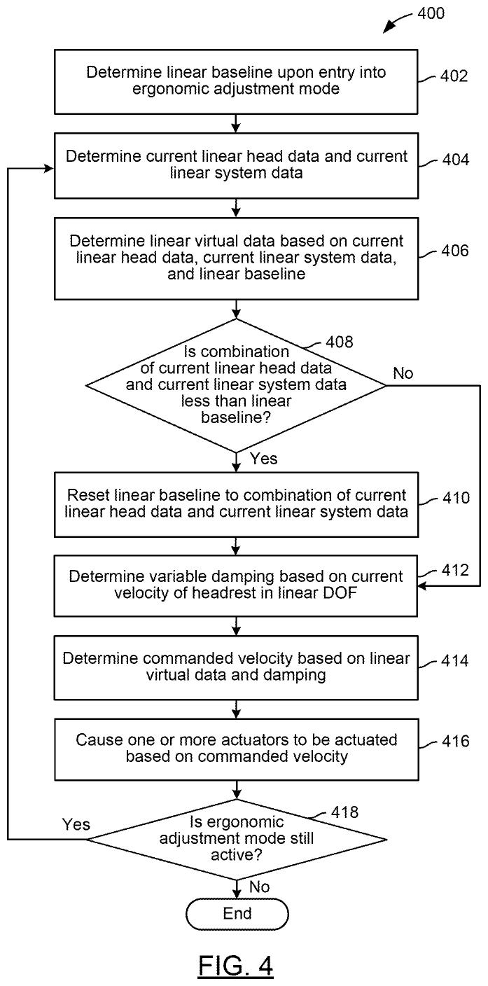

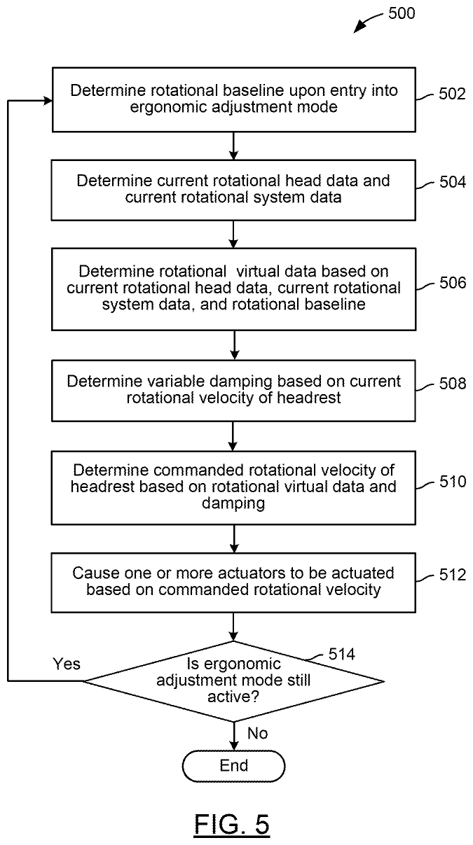

The system data generating module 308 generates system data based on headrest data 304. The headrest data 304 and the system data can be in any number of DOFs, including six or fewer DOFs. In addition, the headrest data 304 and the system data can be represented as vectors, matrices, or in any other suitable manner. The headrest data 304 can be acquired in any technically feasible manner in some embodiments. For example, the headrest data 304 can be computed based on a state of one or more actuators to which the headrest is coupled. As another example, the headrest data 304 can be acquired by one or more sensors. In some embodiments, the headrest data 304 includes a position and/or orientation of the headrest. In such cases, the system data can comprise data based on a virtual spring model that is computed as a function of the position and/or orientation of the headrest. In some other embodiments, the system data can comprise a constant value. In some other embodiments, the system data can comprise a magnitude that is computed according to any technically feasible monotonic function of the position and/or orientation of the headrest, as discussed in greater detail below in conjunction with FIGS. 4-5. In some embodiments, the system data comprises one or more components that are opposite in direction to one or more components of the head data that is generated by the head data module 306. For example, the head data could include a component in an “inward” direction towards a display unit, and the system data could include a component in an “outward” direction away from the display unit (and towards where the operator is expected to be during normal operation). In such cases, the system data is used to generate virtual data that permits the headrest to be adjusted in one or more directions that the head data cannot move the headrest, such as in the “outward” direction away from the display unit.

The virtual data generating module 310 generates the virtual data based on the head data that is output by the head data module 306, the system data that is output by the system data generating module 308, and a baseline. The virtual data can be in any number of DOFs, including six or fewer DOFs, in some embodiments. In addition, the virtual data can be represented as vectors, matrices, or in any other suitable manner. In some embodiments, the virtual data is computed by combining the head data and the system data, and reducing the combination by the baseline. In such cases, combining the head data and the system data can comprise adding the head data (e.g., a head force or torque vector) to the system data (e.g., a system force or torque vector), and reducing the combination using the baseline can comprise subtracting the baseline (e.g., a baselines force or torque vector) from the combination. The baseline is computed by combining head data and system data when the teleoperated system 100 enters the ergonomic adjustment mode. The baseline is used to reduce the combination of the current head data and the current system data, because it is assumed that the baseline at the entry of the ergonomic adjustment mode was not intended by the operator to cause the headrest to move.

The variable damping module 312 generates a commanded velocity 316 by applying a damping to the virtual data that is output by the virtual data generating module 310. In some embodiments, the damping is based on a viscous damping model in which a damping force and/or torque is proportional to a current velocity of the headrest. In such cases, the commanded velocity 316 can be generated by dividing the virtual data by a damping that is based on a current velocity of the headrest (or by multiplying the virtual data by the reciprocal of such a damping). The commanded velocity 316, or one or more commands for achieving the commanded velocity 316, can then be transmitted to one or more actuators that are physically coupled to the headrest, causing the one or more actuators to be actuated so as to move the headrest at the commanded velocity 316.

The ergonomic adjustment activation module 314 detects whether an ergonomic adjustment mode is activated. In some embodiments, the virtual data and the commanded velocity 316, described above, are computed when the ergonomic adjustment mode is activated. The ergonomic adjustment mode can be activated in any technically feasible manner. For example, in some embodiments, the ergonomic adjustment mode can be activated when hand input is detected by one or more hand-input sensors. In such cases, the one or more hand-input sensors can include knobs, finger detectors, joysticks, recessed sensors, among other things. In addition, each hand-input sensor can include strain gauges, touch sensors, springs, etc. and be mounted on a display unit or elsewhere. As another example, in some embodiments, activation of the ergonomic adjustment mode can require a head-present state to be detected. In such cases, the head-present state can require the head of the operator to be within a proximity of the headrest, such as a predefined distance (e.g., 10 cm) from the headrest that is determined based on sensor data acquired by any suitable head-input sensor or sensors. For example, the head-input sensor(s) can include one or more time-of-flight sensors, LIDAR sensors, beam breakers, imaging devices (including monoscopic and stereoscopic optical systems) in conjunction with a computer vision system, ultrasonic systems, depth cameras, or a combination thereof. As yet another example, in some embodiments, activation of the ergonomic adjustment mode can require the hand-input sensors and/or the head-input sensors to pass one or more checks, such as sensor measurements being within expected ranges, multiple (e.g., redundant) sensor measurements being in agreement, etc. As further examples, in some embodiments, the ergonomic adjustment mode can be activated based on input by a control device (e.g., a control device including buttons and/or keys) that the operator interacts with, voice input, input by a user interface, etc.

FIG. 4 illustrates a simplified diagram of a method 400 for adjusting a headrest of a computer-assisted system in a linear DOF, according to various embodiments. One or more of the processes 402-418 of method 400 can be implemented, at least in part, in the form of executable code stored on non-transitory, tangible, machine readable media that when executed by one or more processors (e.g., the processor 150 in control system 140) cause the one or more processors to perform one or more of the processes 402-418. In some embodiments, method 400 can be performed by one or more modules, such as control module 170. In some embodiments, method 400 can include additional processes, which are not shown. In some embodiments, one or more of the processes 402-418 can be performed, at least in part, by one or more of the modules of control system 140.

As shown, the method 400 begins at process 402, where a linear baseline (e.g., a baseline force) is determined upon entry into an ergonomic adjustment mode. Example mechanisms for activating an ergonomic adjustment mode are described above in conjunction with FIG. 3. In some embodiments, the linear baseline can be computed as a combination of linear head data (e.g., a force applied to the headrest by the head of the operator to the headrest) and a linear system data (e.g., a system force) when the ergonomic adjustment mode is first activated. In some embodiments, the linear head data can be determined by setting to zero, or otherwise disregarding, any portions of sensor data (e.g., sensor data 302) in the linear DOF that corresponds to a direction in which the head of an operator cannot move a headrest.

In some embodiments, the linear system data can be a virtual spring force. In such cases, the virtual spring force can comprise a magnitude that is a function of a current position of the headrest in the linear DOF. For example, when the linear DOF is in the inward-outward direction relative to a display unit (e.g., display unit 206), the function can begin at a minimum value when the headrest is in a furthest position in the “outward” direction, remain at the minimum value until the distance of the headrest from the furthest “outward” position is above a first threshold, increase linearly with the distance of the headrest from the furthest “outward” position until a maximum value is reached at a second threshold distance, and remain at the maximum value when the distance of the headrest from the furthest “outward” position is above the second threshold. In such cases, the maximum value is a saturation limit that ensures the linear system data (e.g., a system force) does not become too large. In some embodiments, the first threshold can be zero so that the function begins increasing linearly as soon as the headrest moves away from the furthest “outward” position. In some embodiments, the second threshold can be omitted so that the function continues increasing with the distance of the headrest away from the furthest “outward” position. More generally, in some embodiments, the function can be any technically feasible monotonic function (e.g., a linear function, a piecewise linear function, a quadratic function, and/or the like) of the difference between the headrest position and a predefined position. In some other embodiments, the linear system data can comprise a constant value. For example, in some embodiments, the linear system data can comprise a constant bias force and/or torque.

At process 404, a current linear input (e.g., a current force) applied by the head of the operator to the headrest (“current linear head data”) and a current linear system data (e.g., a current system force) are determined. In some embodiments, the current linear head data is determined based on current sensor data by setting to zero, or otherwise disregarding, any current sensor data in the linear DOF that corresponds to a direction in which the head of the operator cannot move the headrest, similar to the description above in conjunction with process 404. In some embodiments, the current linear system data is determined as a virtual spring force based on the current position of the headrest in the linear DOF, similar to the description above in conjunction with process 402. In some other embodiments, the current linear system data can comprise a constant value, or a value that is determined based on any technically monotonic function of the difference between the current headrest position and a predefined position.

At process 406, linear virtual data (e.g., a virtual force) is determined based on the current linear head data, the current linear system data, and the linear baseline. In some embodiments, the linear virtual data is computed as a combination of the current linear head data and the current linear system data (such as by summing weighted or unweighted data by themselves, which may be further combined with other data), reduced by the linear baseline (e.g. such as by subtracting the linear baseline or a scaled version of the linear baseline, which may be further modified with other data).

At process 408, when a combination of the current linear head data and the current linear system data has a value (e.g., a force magnitude) that is less than a value (e.g., a force magnitude) of the linear baseline, the method continues to process 410, where the value of the linear baseline is reset to the combination of the current linear head data and the current linear system data. That is, the linear baseline is ratcheted down to the combination of the current linear head data and the current linear system data when that combination has a value that is less than a value of the linear baseline.

At process 412, a variable damping is determined based on a current velocity of the headrest in the linear DOF. In some embodiments, the variable damping can comprise a damping force that has a magnitude proportional to a magnitude of the current velocity in the linear DOF up to a saturation limit, after which the magnitude of the damping force remains at a maximum value. In some embodiments, the damping can be different for different directions of the linear DOF. For example, when the linear DOF is in the inward-outward direction relative to the display unit, damping can be greater (e.g., associated with larger damping) for velocities in the “inward” direction than for velocities in the “outward” direction. In such cases, the headrest will move “outward” to meet the head of the operator more quickly than the headrest can be pushed “inward” by the head of the operator.

At process 414, a commanded velocity of the headrest in the linear DOF is determined based on the linear virtual data and the damping. In some embodiments, the commanded velocity can be computed according to a viscous damping model by dividing the linear virtual data by the damping (or multiplying by the reciprocal of such a damping).

At process 416, one or more actuators that are physically coupled to the headrest are actuated based on the commanded velocity. The one or more actuators are operable to move the headrest relative to the display unit. In some embodiments, each actuator can be actuated by transmitting signals, such as voltages, currents, pulse-width modulations, etc. to the actuator.

At process 418, when the ergonomic adjustment mode is still active, the method 400 returns to process 404, where the current linear head data and the current linear system data are determined. Example mechanisms for activating the ergonomic adjustment mode are described above in conjunction with FIG. 3.

Alternatively, when the ergonomic adjustment mode is no longer active, the method 400 ends. For example, the operator can deactivate the ergonomic adjustment mode when the headrest has been adjusted so that the operator has a satisfactory view of images being displayed on the view screen(s) of a display unit, when the operator prefers to not contact the headrest, etc. In some embodiments, the ergonomic adjustment mode is no longer active when hand input is no longer detected by one or more hand-input sensors; a head-present state is no longer detected by one or more head-input sensors; the hand-input sensors and/or the head-input sensors do not pass one or more checks; the operator deactivates the ergonomic adjustment mode by a control device, voice command, or user interface; the computer-assisted system transitions to another mode; a system fault is encountered; the computer-assisted system shuts down; etc.

FIG. 5 illustrates a simplified diagram of a method 500 for adjusting a headrest of a computer-assisted system in a rotational DOF, according to various embodiments. One or more of the processes 502-514 of method 500 can be implemented, at least in part, in the form of executable code stored on non-transitory, tangible, machine readable media that when executed by one or more processors (e.g., the processor 150 in control system 140) can cause the one or more processors to perform one or more of the processes 502-514. In some embodiments, method 500 can be performed by one or more modules, such as control module 170. In some embodiments, method 500 can include additional processes, which are not shown. In some embodiments, one or more of the processes 502-514 can be performed, at least in part, by one or more of the modules of control system 140.

As shown, the method 500 begins at process 502, where a rotational baseline (e.g., a baseline torque) is determined upon entry into an ergonomic adjustment mode. Example mechanisms for activating an ergonomic adjustment mode are described above in conjunction with FIG. 3. In some embodiments, the rotational baseline can be computed as the combination of rotational head data (e.g., a torque applied by the head of the operator to the headrest) and rotational system data (e.g., a system torque) when the ergonomic adjustment mode is first activated. The rotational head data can be determined based on sensor data (e.g., sensor data 302) in the rotational DOF. In contrast to the linear head data, described above in conjunction with FIG. 4, sensor data in the rotational DOF is not set to zero to generate the rotational head data, because the head of an operator can produce torques that rotate a headrest in both directions in each rotation DOF.

In some embodiments, the rotational system data can be a virtual spring torque. In such cases, the virtual spring torque can comprise a magnitude that is a function of a current orientation of the headrest in the rotational DOF. For example, the function can begin at a minimum value when the headrest is at a default orientation when the computer-assisted system (e.g., teleoperated system 100) enters the ergonomic adjustment mode, remain at the minimum value until the headrest is rotated such that a difference between an orientation of the headrest and the default orientation satisfies a first threshold, increase linearly with the difference between the orientation of the headrest and the default orientation until a maximum value is reached when the difference between the orientation of the headrest and the default orientation satisfies a second threshold, and remain at the maximum value when the difference between the orientation of the headrest and the default orientation increases beyond the second threshold. In some embodiments, the first rotation threshold can be zero so that the function begins increasing linearly as soon as the headrest rotates away from the default orientation. In some embodiments, the second threshold can be omitted so that the function continues increasing with the rotation of the headrest away from the default orientation. More generally, in some embodiments, a magnitude of the rotational system data can be determined based on any technically feasible monotonic function (e.g., a linear function, a piecewise linear function, a quadratic function, and/or the like) of the difference between the headrest orientation and a predefined orientation. In some other embodiments, the rotational system data can comprise a constant value.

At process 504, a current rotational input (e.g., a current torque) applied by the head of the operator to the headrest (“current rotational head data”) and a current rotational system data (e.g., a current system torque) are determined. In some embodiments, the current rotational head data is determined based on current sensor data, and the current rotational system data is determined as a virtual spring torque based on a current orientation of the headrest in the rotational DOF, similar to the description above in conjunction with process 502. In some other embodiments, the current rotational system data can comprise a constant value, or a value that is determined based on any technically feasible monotonic function of the difference between the current headrest orientation and a predefined orientation.

At process 506, rotational virtual data (e.g., a virtual torque) is determined based on the current rotational head data, the current rotational system data, and the rotational baseline. In some embodiments, the rotational virtual data is computed as a combination (e.g., a sum) of the current rotational head data and the current rotational system data, reduced by (e.g., minus) the rotational baseline. In contrast to the linear DOF example described above in conjunction with FIG. 4, the rotational baseline is not ratcheted down based on the current rotational input in some embodiments.

At process 508, a variable damping is determined based on a current rotational velocity of the headrest in the rotational DOF. In some embodiments, the variable damping can comprise a damping torque that is proportional to a magnitude of the current rotational velocity of the headrest up to a saturation limit, after which the damping remains at a maximum value. In some embodiments, the damping can be different for different directions of the rotational DOF.

At process 510, a commanded rotational velocity of the headrest in the rotational DOF is determined based on the rotational virtual data and the damping. In some embodiments, the commanded rotational velocity can be computed according to a viscous damping model by dividing the rotational virtual data by the damping (or multiplying by a damping that has an inverse value).

At process 512, one or more actuators that are physically coupled to the headrest are actuated based on the commanded rotational velocity. The one or more actuators are operable to move the headrest relative to the display unit. In some embodiments, each actuator can be actuated by transmitting signals, such as voltages, currents, pulse-width modulations, etc. to the actuator.

At process 514, when the ergonomic adjustment mode is still active, the method 500 returns to process 504, where the current rotational head data and the current rotational system data are determined. Process 514 is similar to process 418, described above in conjunction with FIG. 4. Alternatively, when the ergonomic adjustment mode is no longer active, the method 500 ends.

Although the methods 400 and 500 are described with respect to a single linear DOF and a single rotational DOF, respectively, a headrest can be adjusted in multiple DOF, including up to six DOFs, in some embodiments. For example, in some embodiments, a headrest can be adjusted in an inward-outward direction relative to the display unit. As further examples, in some embodiments, a headrest can also be adjusted in a left-right direction relative to the display unit, an upward-downward direction relative to the display unit, and/or in pitch, yaw, and/or roll (e.g., about the inward-outward direction) rotational directions. In some embodiments, when a headrest is adjustable in multiple DOFs, each DOF is controllable by a separate controller (e.g., a separate controller within control module 170), with the output of each of the separate controllers being superimposed to determine a commanded velocity of the headrest. In some embodiments, when a headrest is adjustable in multiple linear DOFs, the direction of the linear system data (e.g., a virtual spring force) can be towards the location of a forehead of the operator. For example, in some embodiments, the location of the forehead can be determined using eye tracking and/or computer vision techniques, among other things.

Although described herein primarily with respect to adjusting an entire headrest, in some embodiments, methods 400 and 500 can be applied to separately adjust different zones of a headrest when head input at the different zones are measured by head-input sensors. Separately adjusting different zones of the headrest can reshape the headrest in some embodiments.

Although described herein primarily with respect to a headrest of a computer-assisted system, in some embodiments, techniques disclosed herein can be applied to adjust a cushion or rest other than a headrest, and/or different zones thereof. For example, techniques disclosed herein can be applied to adjust a seatback or an arm rest in some embodiments.

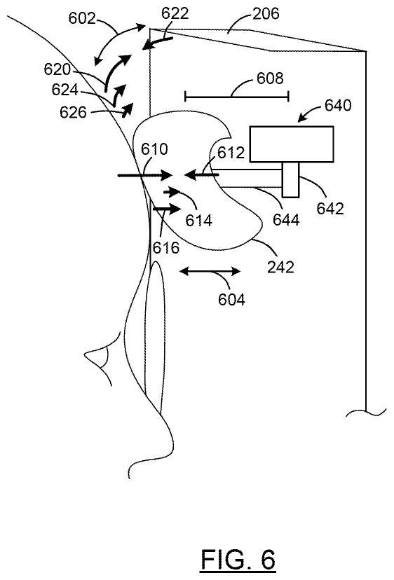

FIG. 6 illustrates an example of controlling a headrest of a display unit, according to various embodiments. As shown, the headrest 242 is adjustable in an inward-outward direction 604 relative to the display unit 206, as well as in a pitch direction 602. In order to determine a commanded velocity of the headrest 242 in the inward-outward direction 604 when an ergonomic adjustment mode is activated, the control module 170 adds linear head data 610 (e.g., a head force) and a linear system data 612 (e.g., a system force). The linear head data 610 can be determined based on sensor data, and the linear system data 612 can be determined based on a position 608 of the headrest 242, as described above in conjunction with FIGS. 3-4. Then, the control module 170 combines the linear head data 610 and the linear system data 612, and reduces the combination by using a linear baseline 614 that is determined when the teleoperated system 100 enters the ergonomic adjustment mode, to obtain linear virtual data 616 (e.g., a virtual force) in the inward-outward direction 604. The control module 170 can combine the linear head data 610 and the linear system data 612 in any appropriate manner, such as by adding unmodified or modified (e.g. scaled) data, and/or by also combining with other data. The control module 170 can reduce the combination by using the linear baseline 614 in any appropriate manner, such as by subtracting unmodified or modified (e.g. scaled) data, and/or by also reducing by other data. In addition, the control module 170 dampens the linear virtual data 616 based on a damping to generate a commanded velocity of the headrest 242 in the inward-outward direction 604. The damping can be determined based on a current velocity of the headrest 242 in the inward-outward direction 604, as described above in conjunction with FIGS. 3-4.

Similarly, in order to determine a commanded rotational velocity of the headrest 242 in the pitch direction 602 when the ergonomic adjustment mode is activated, the control module 170 adds a rotational head data 620 (e.g., a head torque) and a rotational system data 622 (e.g., a system torque). The head data 620 can be determined based on sensor data, and the rotational system data 622 can be determined based on an orientation of the headrest 242, as described above in conjunction with FIGS. 3 and 5. Then, the control module 170 combines (e.g., adds together or other combination technique) the rotational head data 620 and the rotational system data 622, and reduces (e.g., subtracts from or other reduction technique) the combination by a rotational baseline 624 that is determined when the teleoperated system 100 enters the ergonomic adjustment mode, to generate rotational virtual data 624 (e.g., a virtual torque) in the pitch direction 602. In addition, the control module 170 dampens the rotational virtual data 626 based on a damping to generate a commanded rotational velocity of the headrest 242 in the pitch direction 602. The damping can be determined based on a rotational velocity of the headrest 242 in the pitch direction 602, as described above in conjunction with FIGS. 3 and 5.

Thereafter, the control module 170 causes an actuator 640 to be actuated based on the commanded velocity in the inward-outward direction 604 and the commanded rotational velocity in the pitch direction 602. Illustratively, the actuator 640 is physically coupled to move the headrest 242 and, in particular, is configured to move/adjust the position of headrest 242 in the inward-outward direction 604 relative to the display unit 206 as well as in the pitch direction 602. In operation, the actuator 640 can be controlled by any technically feasible control system, such as the control module 170, and/or operator input to move the headrest 242. In some embodiments, the control system and/or operator input devices can communicate, directly or indirectly, with an encoder (not shown) included in the actuator 640 to cause a motor to rotate a ball screw (not shown). As the ball screw rotates, a ball screw nut (not shown) that is coupled to a sled 642 moves along the inward-outward direction 604 on a rail (not shown). The sled 642 is, in turn, coupled to a shaft 644 of the headrest 242 and slidably connected to the rail. Accordingly, the headrest 242 is moved along the inward-outward direction 604. In addition, the actuator 640 can include any technically feasible mechanism to pivot the sled 642, or the entire actuator 640, about an appropriate axis to move the headrest 242 along the pitch direction 602. In some embodiments, other mechanisms can be employed to adjust/move a headrest of a display unit in accordance with the present disclosure. For example, other electromechanical, or one or more mechanical, hydraulic, pneumatic, or piezoelectric actuators can be employed to move an adjustable headrest of a display unit in accordance with this disclosure. As examples, a geared actuator or a kinematic mechanism/linkage could be employed to move the headrest 242. Additional examples of moveable display systems are described in concurrently filed U.S. Provisional Patent Application having attorney docket number P06424-US-PRV and entitled “Adjustable Headrest for a Display Unit of a Teleoperated Surgical System,” which is incorporated by reference herein.

Accordingly, in an example, an operator can adjust the headrest 242 by (1) activating the ergonomic adjustment mode; (2) applying a head input to command (such as by pushing and/or torquing with the head) the headrest to a desired position and/or orientation, or reducing a magnitude of a head input so that system data causes an actuator to move the headrest toward the operator and “follow” the head of the operator to a desired position and/or orientation; and (3) disabling the ergonomic adjustment mode when the headrest 242 is in the desired position and/or orientation. Further, use of variable damping to reduce a velocity of the headrest 242 during the adjustment, as described above in conjunction with FIGS. 3-5, causes the headrest 242 to mimic the compression of a pillow that becomes firmer when the operator presses his or head against the headrest 242, and the expansion of a pillow when the operator releases his or head from the headrest 242.

The disclosed techniques can reposition a headrest relative to a display unit of a computer-assisted system. Such a repositioning can result in greater operator comfort and/or permit greater operator comfort and/or the operator to see an entire image being displayed by the display unit of the computer-assisted system and/or to see a properly fused image that combines images seen through different lenses, when the display unit includes lenses. Further, operator discomfort, eye fatigue, etc. can be avoided or reduced.

Some examples of control systems, such as control system 140 may include non-transitory, tangible, machine readable media that include executable code that when executed by one or more processors (e.g., processor 150) may cause the one or more processors to perform the processes of methods 400 and/or 500 and/or the processes of FIGS. 4 and/or 5. Some common forms of machine readable media that may include the processes of methods 400 and/or 500 and/or the processes of FIGS. 4 and/or 5 are, for example, floppy disk, flexible disk, hard disk, magnetic tape, any other magnetic medium, CD-ROM, any other optical medium, punch cards, paper tape, any other physical medium with patterns of holes, RAM, PROM, EPROM, FLASH-EPROM, any other memory chip or cartridge, and/or any other medium from which a processor or computer is adapted to read.

Although illustrative embodiments have been shown and described, a wide range of modification, change and substitution is contemplated in the foregoing disclosure and in some instances, some features of the embodiments may be employed without a corresponding use of other features. One of ordinary skill in the art would recognize many variations, alternatives, and modifications. Thus, the scope of the invention should be limited only by the following claims, and it is appropriate that the claims be construed broadly and, in a manner, consistent with the scope of the embodiments disclosed herein.

What is claimed is:

1. A computer-assisted system comprising:
a display unit configured to display images viewable by an operator;
a headrest coupled to the display unit, the headrest configured to be contacted by a head of the operator;
an actuator operable to move the headrest relative to the display unit;
a head-input sensor; and
a control unit communicably coupled to the actuator and the head-input sensor,
wherein the control unit is configured to:
determine head data based on sensor data acquired by the head-input sensor,
determine a commanded motion based on at least the head data, a baseline, and a damping, and
command the actuator to move the headrest based on the commanded motion.

2. The computer-assisted system of claim 1, wherein the control unit is further configured to determine the commanded motion based on a virtual spring model and at least one parameter selected from the group consisting of: a system force or a system torque.

3. The computer-assisted system of claim 2, wherein the control unit is further configured to determine the commanded motion based on at least one parameter selected from the group consisting of: a position of the headrest and an orientation of the headrest.

4. The computer-assisted system of claim 2, wherein the control unit is further configured to determine the commanded motion based on a monotonic function of a difference between a headrest position of the headrest and a predefined position.

5. The computer-assisted system of claim 4, wherein the monotonic function outputs a first value when a value of the difference is below a first threshold, increases from the first value to a second value when the value of the difference increases from the first threshold to a second threshold, and has the second value when the value of the difference is above the second threshold.

6. The computer-assisted system of claim 2, wherein the control unit is further configured to determine the commanded motion based on a monotonic function of a difference between a headrest orientation of the headrest and to a predefined orientation.

7. The computer-assisted system of claim 1, wherein the control unit is further configured to determine the damping based on a velocity of the headrest.

8. The computer-assisted system of claim 7, wherein the damping varies based on a direction of the velocity.

9. The computer-assisted system of claim 8, wherein a value of the damping is larger when the velocity is in a first direction than when the velocity is in a second direction.

10. The computer-assisted system of claim 9, wherein the velocity in the first direction moves the headrest towards the display unit, and wherein the velocity in the second direction moves the headrest away from the display unit.

11. The computer-assisted system of claim 1, wherein the control unit is further configured to determine the baseline based on:
first head data determined based on the sensor data acquired by the head-input sensor when the computer-assisted system enters an ergonomic adjustment mode; and
first system data associated with when the computer-assisted system enters the ergonomic adjustment mode.

12. The computer-assisted system of claim 1, wherein the head data includes at least one parameter selected from the group consisting of: a force associated with the head, a position of the head, a torque associated with the head, and an orientation of the head.

13. The computer-assisted system of claim 1, further comprising:
another actuator communicably coupled to the control unit,
wherein to command the actuator to move the headrest based on the commanded motion, the control unit is configured to:
command the actuator to move a portion of the headrest; and
command the another actuator to move another portion of the headrest.

14. A method for controlling a headrest coupled to a display unit of a computer-assisted system, the headrest configured to be contacted by a head of an operator, and the display unit configured to display images viewable by the operator, the method comprising:
determining head data based on sensor data acquired by a head-input sensor,
determining a commanded motion based on at least the head data, a baseline, and a damping, and
commanding an actuator to move the headrest based on the commanded motion, wherein the actuator is operable to move the headrest relative to the display unit.

15. The method of claim 14, wherein determining the commanded motion is further based on a virtual spring model and at least one parameter selected from the group consisting of: a system force or a system torque.

16. The method of claim 15, wherein determining the commanded motion is further based on at least one parameter selected from the group consisting of: a position of the headrest and an orientation of the headrest.

17. The method of claim 15, wherein determining the commanded motion is further based on a monotonic function of a difference between a headrest position of the headrest and a predefined position.

18. The method of claim 14, further comprising determining the damping based on a velocity of the headrest, wherein the damping varies based on a direction of the velocity.

19. The method of claim 14, further comprising determining the baseline based on:
first head data determined based on the sensor data acquired by the head-input sensor when the computer-assisted system enters an ergonomic adjustment mode; and
first system data associated with when the computer-assisted system enters the ergonomic adjustment mode.

20. One or more non-transitory machine-readable media comprising a plurality of machine-readable instructions which when executed by one or more processors are adapted to cause the one or more processors to perform a method for controlling a headrest coupled to a display unit of a computer-assisted system, the headrest configured to be contacted by a head of an operator, and the display unit configured to display images viewable by the operator, the method comprising:
determining head data based on sensor data acquired by a head-input sensor,
determining a commanded motion based on at least the head data, a baseline, and a damping, and
commanding an actuator to move the headrest based on the commanded motion, wherein the actuator is operable to move the headrest relative to the display unit.
